(19) United States
(12) Patent Application Publication
(10) Pub. No.: US 2025/0107793 A1
(43) Pub. Date: Apr. 3, 2025

(54) SURGICAL LOCKING BUTTONS

(71) Applicant: ARTHREX, INC., Naples, FL (US)

(72) Inventors: Peter J. DREYFUSS, Naples, FL (US); Jacob A. JOLLY, Naples, FL (US)

(21) Appl. No.: 18/420,830

(22) Filed: Jan. 24, 2024

Related U.S. Application Data
(60) Provisional application No. 63/586,071, filed on Sep. 28, 2023.

(51) Int. Cl. A61B 17/04

(52) U.S. Cl. CPC A61B 17/0401; A61B 2017/0404; A61B 2017/0445; A61B 2017/0456; A61B 2017/0462

(57) ABSTRACT

Graft fixation systems and methods are provided for securing a graft within a bone tunnel. An exemplary graft fixation system may include a surgical locking button adapted for fixating both a graft and a reinforcement suture relative to bone.

### CROSS-REFERENCE TO RELATED APPLICATIONS

This application claims the benefit of U.S. Provisional Application No. 63/586,071, which was filed on Sep. 28, 2023 and is incorporated herein by reference in its entirety.

### BACKGROUND

This disclosure relates to the field of surgery, and more particularly to graft fixation systems that include a surgical locking button adapted to establish a common point of fixation for a graft and a reinforcement suture that is separate from the graft.

Tissue reconstruction surgeries, such as anterior cruciate ligament (ACL) reconstructions and posterior cruciate ligament (PCL) reconstructions, typically involve drilling a tunnel through bone, positioning a substitute graft in the bone tunnel, and fixating the graft within the bone tunnel using one or more fixation devices, such as buttons, screws, or the like.

### SUMMARY

This disclosure relates to graft fixation systems and methods for securing a graft within a bone tunnel. The graft fixation system can be used in various tissue reconstruction procedures, including but not limited to, ACL and PCL reconstructions, for example.

An exemplary surgical locking button may include, inter alia, a disk portion, a barrel portion protruding from the disk portion, a cannulation extending through the disk portion and the barrel portion, a pair of apertures extending through the disk portion and configured for securing a graft relative to a bone, and a locking barb provided within the cannulation and configured for locking a reinforcement suture relative to the bone.

An exemplary surgical method may include, inter alia, loading a reinforcement suture through a cannulation of a surgical locking button, positioning the surgical locking button relative to a bone tunnel of a bone, fixating a graft within the bone tunnel with the surgical locking button, tensioning the reinforcement suture in a first direction, and locking the reinforcement suture relative to the surgical locking button to prevent movement in a second direction.

Another exemplary surgical method may include, inter alia, positioning a surgical locking button relative to a bone tunnel of a bone. The surgical locking button fixates both a graft and a reinforcement suture relative to the bone.

The embodiments, examples, and alternatives of the preceding paragraphs, the claims, or the following description and drawings, including any of their various aspects or respective individual features, may be taken independently or in any combination. Features described in connection with one embodiment are applicable to all embodiments, unless such features are incompatible.

The various features and advantages of this disclosure will become apparent to those skilled in the art from the following detailed description. The drawings that accompany the detailed description can be briefly described as follows.

### BRIEF DESCRIPTION OF THE DRAWINGS

FIG. 1 illustrates a graft fixation system for securing a graft within a bone tunnel.

FIG. 2 illustrates an exemplary reinforcement suture of the graft fixation system of FIG. 1.

FIG. 3 illustrates an exemplary surgical locking button of the graft fixation system of FIG. 1.

FIG. 4 is a cross-sectional view through section 4-4 of FIG. 3.

FIG. 5 illustrates another exemplary surgical locking button.

FIG. 6A is a blown-up view of region 6 of FIG. 3.

FIG. 6B illustrates a plurality of rows of locking barbs of the surgical locking button of FIG. 2.

FIG. 7 schematically illustrates an exemplary use of the surgical locking button of FIGS. 3-4 for fixating both a graft and a reinforcement suture.

FIGS. 8 and 9 illustrate another exemplary surgical locking button for a graft fixation system.

### DETAILED DESCRIPTION

This disclosure relates to graft fixation systems and methods for securing a graft within a bone tunnel. The graft fixation systems may include a surgical button, a loop connected to the surgical button, a graft carried by the loop, a reinforcement suture connected to the button and tensionable separately from the graft, and a surgical locking button. The graft fixation system can be used in various tissue reconstruction procedures, including but not limited to, ACL and PCL reconstructions, for example. These and other features of this disclosure are described in further detail below.

An exemplary surgical locking button may include, inter alia, a disk portion, a barrel portion protruding from the disk portion, a cannulation extending through the disk portion and the barrel portion, a pair of apertures extending through the disk portion and configured for securing a graft relative to a bone, and a locking barb provided within the cannulation and configured for locking a reinforcement suture relative to the bone.

In any further embodiment, the disk portion and the barrel portion establish a unitary body of the surgical locking button.

In any further embodiment, the locking barb is an integral feature of the unitary body.

In any further embodiment, the locking barb is part of a locking ferrule that is received within the cannulation.

In any further embodiment, the locking barb includes a pointed tip that is configured to engage the reinforcement suture.

In any further embodiment, the disk portion includes a top surface, a bottom surface, and a side wall extending between the top surface and the bottom surface.

In any further embodiment, the locking barb is angled in a direction toward the top surface.

In any further embodiment, the barrel portion protrudes from the bottom surface.

In any further embodiment, each aperture of the pair of apertures is a round hole.

In any further embodiment, each aperture of the pair of apertures is a slot that extends through a side wall of the disk portion.

An exemplary surgical method may include, inter alia, loading a reinforcement suture through a cannulation of a surgical locking button, positioning the surgical locking button relative to a bone tunnel of a bone, fixating a graft within the bone tunnel with the surgical locking button, tensioning the reinforcement suture in a first direction, and locking the reinforcement suture relative to the surgical locking button to prevent movement in a second direction.

In any further embodiment, the reinforcement suture is separate from and unattached to the graft.

In any further embodiment, a locking barb protrudes inwardly from an interior wall of the surgical locking button.

In any further embodiment, the locking barb is part of a locking ferrule that is received within the cannulation.

In any further embodiment, the surgical method includes inserting the locking ferrule into the cannulation prior to loading and tensioning the reinforcement suture.

In any further embodiment, the locking barb engages the reinforcement suture to prevent movement in the second direction.

In any further embodiment, the locking barb interdigitates with a thickened portion of the reinforcement suture to prevent movement in the second direction.

Another exemplary surgical method may include, inter alia, positioning a surgical locking button relative to a bone tunnel of a bone. The surgical locking button fixates both a graft and a reinforcement suture relative to the bone.

In any further embodiment, the graft is secured to the surgical locking button by a suture.

In any further embodiment, a locking barb of the surgical locking button locks a position of the reinforcement suture relative to the surgical locking button.

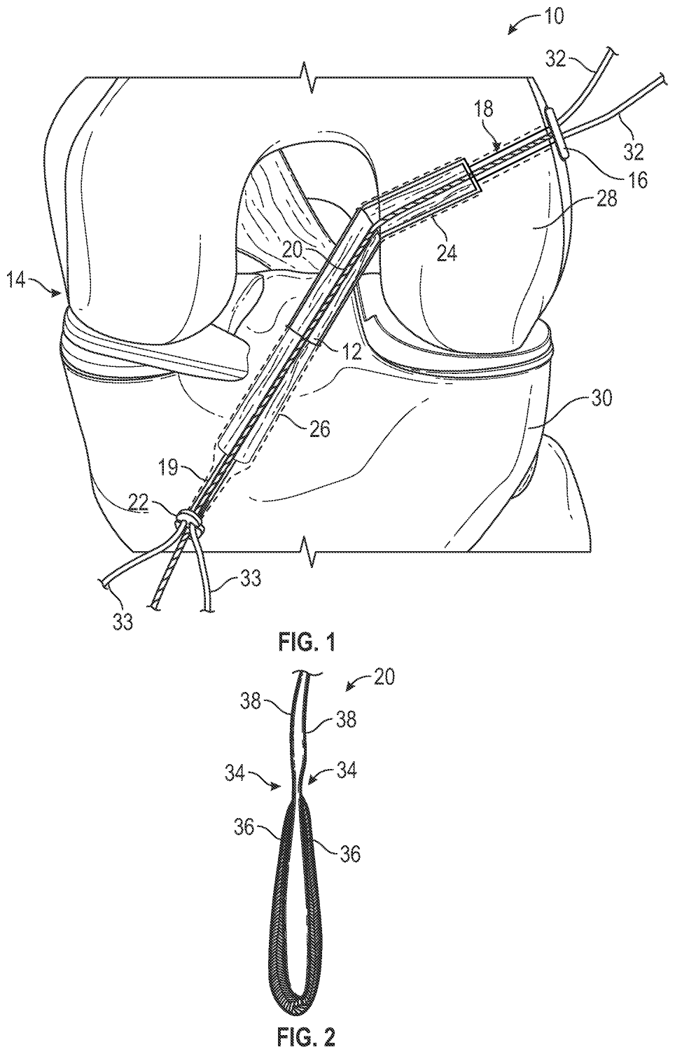

FIG. 1 illustrates an exemplary graft fixation system 10 for fixating a graft 12 within a joint 14 (e.g., a knee joint). The graft fixation system 10 may be used to fixate the graft 12 when performing a variety of tissue reconstruction procedures, for example. The tissue reconstruction procedures could include any surgical procedure in which it is desirable to position a replacement graft within a bone tunnel to repair torn tissue. ACL and PCL reconstructions are but two non-limiting examples of surgical reconstruction procedures that could benefit from the use of the graft fixation systems 10 of this disclosure. Other surgical procedures that could benefit from the teachings of this disclosure include but are not limited to acromioclavicular (AC) joint repairs.

The graft fixation system 10 may be implanted within the joint 14 to repair a torn tissue (e.g., a torn ACL). Prior to implanting the graft fixation system 10 within the joint 14, a first bone tunnel 24 (e.g., a socket) may be formed in a first bone 28 (e.g., a femur), and a second bone tunnel 26 (e.g., a passage) may be formed in a second bone 30 (e.g., a tibia).

The graft fixation system 10 may include, among various other components or features, a surgical button 16, a first loop 18, a second loop 19, a reinforcement suture 20, and a surgical locking button 22. The surgical button 16 may be configured to provide cortical bone fixation of the graft 12 relative to the first bone 28 after the graft 12 has been positioned within the first bone tunnel 24. The surgical button 16 may include one or more apertures formed through the body of the surgical button 16 for receiving and carrying both the first loop 18 and the reinforcement suture 20. The surgical button 16 may be oblong or round and may be made of either metallic or polymeric materials within the scope of this disclosure.

The first loop 18 may be an adjustable loop made of a flexible material (e.g., suture) and includes an adjustable length and perimeter. Tensioning strands 32 of the first loop 18 may be pulled to reduce the size of the first loop 18. For example, the first loop 18 may be reduced in size by pulling the tensioning stands 32 in a first direction but is prevented from loosening in the opposite direction due to applied internal tensile forces. The tensioning strands 32 may be spliced through portions of the first loop 18 for establishing splices that are configured to constrict when tensioned to create the applied internal tensile forces.

The second loop 19 may also be an adjustable loop made of a flexible material (e.g., suture) and includes an adjustable length and perimeter. Tensioning strands 33 of the second loop 19 may be pulled to reduce the size of the second loop 19. For example, the second loop 19 may be reduced in size by pulling the tensioning stands 33 in a first direction but is prevented from loosening in the opposite direction due to applied internal tensile forces. The tensioning strands 33 may be spliced through portions of the second loop 19 for establishing splices that are configured to constrict when tensioned to create the applied internal tensile forces.

The first loop 18 may connect to the surgical button 16, and the second loop 19 may connect to the surgical locking button 22. The graft 12 may extend between the first loop 18 and the second loop 19 and may therefore be connected to the surgical button 16 and the surgical locking button 22 by the first loop 18 and the second loop 19, respectively. The graft 12 may be folded or looped over a cradle portion of each of the first loop 18 and the second loop 19. Alternatively, the graft 12 could connected to the surgical locking button 22 by a suture that is whipstitched to the graft 12, such as by tying a knot over top of the surgical locking button, for example.

The reinforcement suture 20 may be connected to the surgical button 16. For example, the reinforcement suture 20 may be received through apertures formed through the surgical button 16. The reinforcement suture 20 is therefore unconnected to the graft 12 and can be tensioned separately from the graft 12. For example, the graft 12 and the reinforcement suture 20 can be tensioned separately from one another, resulting in independent tension loads. Tension can be applied to the graft 12 (e.g., by tensioning the first loop 18 and/or the second loop 19) after tensioning the reinforcement suture 20. Accordingly, joint loads may be shared between the graft 12 and the reinforcement suture 20, with the reinforcement suture 20 acting as a dynamic joint stabilizer that shares loads with and reinforces the primary repair provided by the graft 12. The reinforcement suture 20 may therefore be referred to as an “internal brace” or “safety belt” feature of the graft fixation system 10.

The reinforcement suture 20 may include one or more strands of suture tape, such as FiberTape®, for example. FiberTape® is a suture product marketed and sold by Arthrex, Inc. However, other suture products could be utilized for the reinforcement suture 20 within the scope of this disclosure.

The reinforcement suture 20 may include a varying thickness. The reinforcement suture 20 may therefore include one or more tapered regions 34 where the reinforcement suture 20 transitions between a thickened section 36 and a thinned section 38 (see, e.g., FIG. 2). The thinned sections 38 can facilitate passing the reinforcement suture 20 through the cannulation 54, such as via a suitable suture loader that can be provided as part of a suture locking system that includes the surgical locking button 22, for example, and the thickened sections 36 can provide greater surface area for the locking barbs 58 to engage in order to sufficiently lock the reinforcement suture 20 relative to the surgical locking button 22.

In an embodiment, the thickened sections 36 of the reinforcement suture 20 are about twice as thick as the thinned sections 38. However, other ratios (e.g., 1.5:1, 3:1, etc.) between the relative thicknesses (e.g., outer diameters) of the thickened sections 36 and the thinned sections 38 are contemplated within the scope of this disclosure.

In an embodiment, the graft fixation system 10 may be implanted within the joint 14 by passing the surgical button 16 through the first bone tunnel 24. The surgical button 16 may be pulled through the first bone tunnel 24 using a passing suture (not shown) and can self-flip onto the cortex of the first bone 28 once tension is released on the passing suture. Alternatively, a flipping suture (not shown) could be used to seat the surgical button 16 relative to the cortex.

After passing and flipping the surgical button 16, the first loop 18 may suspend the graft 12 within the first bone tunnel 24. The tensioning strands 32 of the first loop 18 can be pulled to adjust the size of the first loop 18 and to fully seat the graft 12 within the first bone tunnel 24.

The graft 12, the second loop 19, and the reinforcement suture 20 may subsequently be arranged to extend within the second bone tunnel 26. The tensioning strands 33 of the second loop 19 can be pulled to adjust the size of the second loop 19 and to fully seat the graft 12 within the second bone tunnel 26.

The graft 12 and the reinforcement suture 20 can be separately tensioned prior to completing the technique. For example, a first tension can be applied to the graft 12 by applying traction to the tensioning strands 33 of the second loop 19 (or to sutures that are whipstitched to the graft 12), and a second, different tension can be applied by applying traction to the reinforcement suture 20 since these components are unconnected to one another. The graft 12 can thus be retensioned after intraoperative preconditioning. Intraoperative preconditioning can be used to reduce residual laxity in the graft 12. Graft retensioning optimizes the mechanical stability of soft tissue by decreasing its dynamic elongation. The reinforcement suture 20 may provide increased stiffness compared to soft tissue material, thus its resistance to dynamic elongation may be high enough without retensioning.

The graft 12 and the reinforcement suture 20 can both be fixated relative to the second bone 30 by the surgical locking button 22. In prior techniques, a separate screw or anchor was typically necessary to fixate the reinforcement suture 20 when securing the graft 12 relative to the second bone 30. However, as further discussed below, the surgical locking button 22 may be specifically configured to provide a common point of fixation for fixating both the graft 12 and the reinforcement suture 20 relative to the second bone 30.

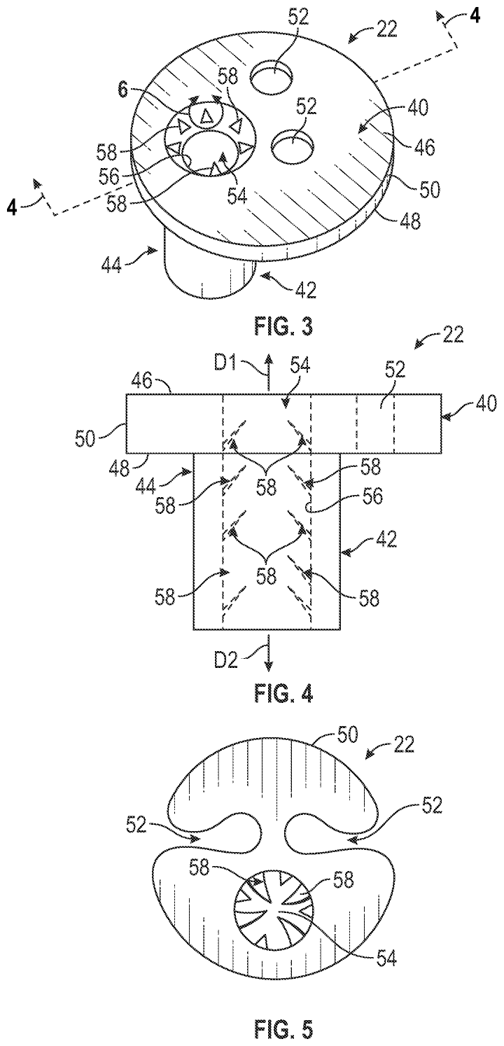

FIGS. 3 and 4 illustrate an exemplary surgical locking button 22 that can be utilized as part of the graft fixation system 10 described above. The surgical locking button 22 may include a disk portion 40 and a barrel portion 42 that protrudes from the disk portion 40. The disk portion 40 and the barrel portion 42 may be formed together to establish a unitary body 44 of the surgical locking button 22.

The disk portion 40 of the surgical locking button 22 may include a top surface 46, a bottom surface 48, and a side wall 50 that extends between the top surface 46 and the bottom surface 48. The side wall 50 may be a circumferential surface for establishing the disk-like shape of the disk portion 40.

A pair of apertures 52 may be formed through the disk portion 40 and therefore extend through both the top surface 46 and the bottom surface 48. The apertures 52 can be used to connect the second loop 19 (or sutures that are whipstitched to the graft 12) to the surgical locking button 22. The apertures 52 may be configured as either round holes (see FIG. 3) or slots that open through the side wall 50 of the disk portion 40 (see FIG. 5).

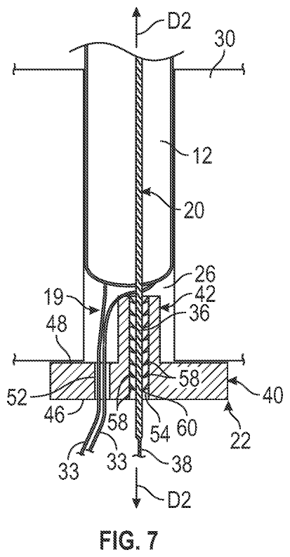

A cannulation 54 may extend through the disk portion 40 and the barrel portion 42. The cannulation 54 may establish an internal passageway for accommodating the reinforcement suture 20 (see, for example, FIG. 7) or other structures (e.g., wires, shafts, etc.).

The cannulation 54 may be circumscribed by an interior wall 56 of the unitary body 44. The interior wall 56 may be part of both the disk portion 40 and the barrel portion 42. The cannulation 54 may be straight or may taper in a direction toward a tip of the barrel portion 42.

A plurality of locking barbs 58 may protrude inwardly from the interior wall 56. The locking barbs 58 may therefore occupy at least a portion of the open space of the cannulation 54. In an embodiment, the locking barbs 58 are integrally formed (e.g., molded) features of the unitary body 44. The locking barbs 58 may be provided along an entire length of the cannulation 54 or at select portions thereof. The locking barbs 58 may be either rigid or flexible structures.

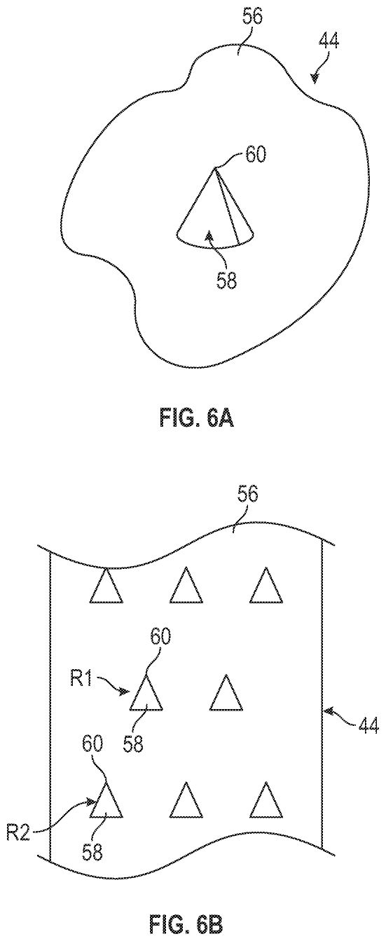

Each locking barb 58 may include a sharp or pointed tip 60 (see FIG. 6A), and each locking barb 58 may be angled in a direction toward the top surface 46 of the disk portion 40. The locking barbs 58 may therefore establish a one-way locking mechanism that permits a structure, such as the reinforcement suture 20, to pass through the cannulation 54 in a first direction D1 while preventing the structure from being tensioned or otherwise moved in a second direction D2.

The locking barbs 58 may be arranged in multiple rows along the length of the cannulation 54. For example, the locking barbs 58 may be arranged in a least a first row R1 and a second row R2 (see FIG. 6B). In an embodiment, the locking barbs 58 of the second row R2 are staggered relative to the locking barbs 58 of the first row R1 (see FIG. 6B).

Referring now to FIG. 7, with continued reference to FIGS. 1-6, the surgical locking button 22 may positioned relative to the second bone 30 such that the barrel portion 42 is accommodated within the second bone tunnel 26 and the disk portion 40 is seated relative to the cortex of the second bone 30. In the seated position, the top surface 46 of the disk portion 40 faces away from the second bone 30 and the bottom surface 48 faces toward the second bone 30. The reinforcement suture 20 may be received through the cannulation 54 prior to positioning the surgical locking button 22 relative to the second bone 30. For example, one or more thinned sections 38 of the reinforcement suture 20 may be passed through the cannulation 54 by pulling the thinned sections 38 in the first direction D1.

Once the reinforcement suture 20 has been passed through the cannulation 54 of the surgical locking button 22 and the surgical locking button 22 has been seated relative to the second bone tunnel 26 of the second bone 30, the reinforcement suture 20 may be tensioned in the first direction D1 to lock the reinforcement suture 20 relative to the second bone 30. The locking barbs 58 prevent the reinforcement suture 20 from backing up or otherwise moving in the second direction D2. For example, if tension is applied in the second direction D2, the pointed tips 60 of the locking barbs 58 may interdigitate with one or more of the thickened sections 36 of the reinforcement suture 20 and thereby prevent it from moving in the second direction D2. The surgical locking button 22 is therefore equipped to provide the dual function of both fixating the graft 12 and the reinforcement suture 20 relative to the second bone 30.

Although shown as locking a single reinforcement suture 20 in the above implementations, the surgical locking button 22 could be configured to receive and lock multiple strands of suture.

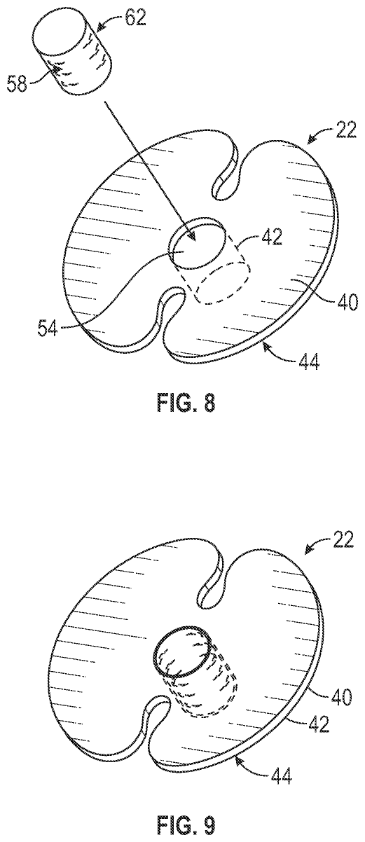

In the above embodiment, the locking barbs 58 are integral features of the unitary body 44 of the surgical locking button 22. However, other implementations are contemplated within the scope of this disclosure. For example, as shown in FIGS. 8-9, the locking barbs 58 could be integral features of a separate locking ferrule 62 that is insertable into the cannulation 54 of the surgical locking button 22. The locking ferrule 62 may be secured to the unitary body 44 of the surgical locking button 22 via an interference fit. The locking ferrule 62 can be accommodated within the cannulation 54 either during manufacturing (e.g., via an overmolding process) or while performing the surgical reconstruction procedure (such as, for example, just prior to inserting the surgical locking button 22 into a bone tunnel). Inserting the locking ferrule 62 during the surgical procedure can simplify suture handling and prevent the premature locking of the reinforcement suture 20.

The graft fixation systems and surgical methods described herein may be utilized to secure grafts within bone tunnels. The graft fixation systems may include surgical locking buttons that include internal one-way locking mechanisms. Accordingly, a single fixation device can be utilized for achieving fixation of both a graft and a reinforcement suture that is separate from and unattached to the graft.

Although the different non-limiting embodiments are illustrated as having specific components or steps, the embodiments of this disclosure are not limited to those particular combinations. It is possible to use some of the components or features from any of the non-limiting embodiments in combination with features or components from any of the other non-limiting embodiments.

It should be understood that like reference numerals identify corresponding or similar elements throughout the several drawings. It should further be understood that although a particular component arrangement is disclosed and illustrated in these exemplary embodiments, other arrangements could also benefit from the teachings of this disclosure.

The foregoing description shall be interpreted as illustrative and not in any limiting sense. A worker of ordinary skill in the art would understand that certain modifications could come within the scope of this disclosure. For these reasons, the following claims should be studied to determine the true scope and content of this disclosure.

What is claimed is:

1. A surgical locking button, comprising:
a disk portion;
a barrel portion protruding from the disk portion;
a cannulation extending through the disk portion and the barrel portion;
a pair of apertures extending through the disk portion and configured for securing a graft relative to a bone; and
a locking barb extending into the cannulation and configured for locking a reinforcement suture relative to the bone.

2. The surgical locking button as recited in claim 1, wherein the disk portion and the barrel portion establish a unitary body of the surgical locking button.

3. The surgical locking button as recited in claim 2, wherein the locking barb is an integral feature of the unitary body.

4. The surgical locking button as recited in claim 1, wherein the locking barb is part of a locking ferrule that is received within the cannulation.

5. The surgical locking button as recited in claim 1, wherein the locking barb includes a pointed tip that is configured to engage the reinforcement suture.

6. The surgical locking button as recited in claim 1, wherein the disk portion includes a top surface, a bottom surface, and a side wall extending between the top surface and the bottom surface.

7. The surgical locking button as recited in claim 6, wherein the locking barb is angled in a direction toward the top surface.

8. The surgical locking button as recited in claim 6, wherein the barrel portion protrudes from the bottom surface.

9. The surgical locking button as recited in claim 1, wherein each aperture of the pair of apertures is a round hole.

10. The surgical locking button as recited in claim 1, wherein each aperture of the pair of apertures is a slot that extends through a side wall of the disk portion.

11. A surgical method, comprising:
loading a reinforcement suture through a cannulation of a surgical locking button;
positioning the surgical locking button relative to a bone tunnel of a bone,
fixating a graft within the bone tunnel with the surgical locking button;
tensioning the reinforcement suture in a first direction; and
locking the reinforcement suture relative to the surgical locking button to prevent movement in a second direction.

12. The surgical method as recited in claim 11, wherein the reinforcement suture is separate from and unattached to the graft.

13. The surgical method as recited in claim 11, wherein a locking barb protrudes inwardly from an interior wall of the surgical locking button.

14. The surgical method as recited in claim 13, wherein the locking barb is part of a locking ferrule that is received within the cannulation.

15. The surgical method as recited in claim 14, comprising:
inserting the locking ferrule into the cannulation prior to loading and tensioning the reinforcement suture.

16. The surgical method as recited in claim 13, wherein the locking barb engages the reinforcement suture to prevent movement in the second direction.

17. The surgical method as recited in claim 16, wherein the locking barb interdigitates with a thickened portion of the reinforcement suture to prevent movement in the second direction.

18. A surgical method, comprising:
positioning a surgical locking button relative to a bone tunnel of a bone,
wherein the surgical locking button fixates both a graft and a reinforcement suture relative to the bone.

19. The surgical method as recited in claim 18, wherein the graft is secured to the surgical locking button by a suture.

20. The surgical method as recited in claim 18, wherein a locking barb of the surgical locking button locks a position of the reinforcement suture relative to the surgical locking button.
